(19) United States
(12) Patent Application Publication
(10) Pub. No.: US 2023/0032147 A1
(43) Pub. Date: Feb. 2, 2023

(54) AUDIOMETRIC SYSTEM AND METHOD WITH AMBIENT NOISE DISTRACTION AND MASKING INDENTIFICATION

(71) Applicant: Audiology Incorporated, Arden Hills, MN (US)

(72) Inventors: Robert H. Margolis, Arden Hills, MN (US); George L. Saly, Canmore (CA)

(21) Appl. No.: 17/387,109

(22) Filed: Jul. 28, 2021

(51) Int. Cl. A61B 5/12; H04R 1/10; A61B 5/00

(52) U.S. Cl. CPC A61B 5/123; H04R 1/1008; A61B 5/7221; A61B 2560/0247

(57) ABSTRACT

A system and method for testing hearing of a subject in an environment in which ambient noise is present, which identifies threshold levels that require retesting due to presence of masking or distraction created by ambient noise during pure tone audiometry. An acoustic sensor monitors ambient noise in the vicinity of the subject during pure tone audiometry while pure tone stimuli are presented.

### BACKGROUND

Pure tone audiometry is a hearing test that is used to determine hearing loss of a person being tested (“the subject”). Typically, the subject is presented a series of pure tone stimuli at specific frequencies in the range from 250 Hertz (Hz) and 8 kHz. For high frequency audiometry, pure tone stimuli between 8 kHz and 16 kHz are used. The intensity (or decibel level) of the pure tone stimulus is varied incrementally, and the subject indicates when he or she hears the pure tone stimulus. A Threshold Level is determined for each stimulus frequency. A Criterion Level is a level corresponding to a “Yes” response by the subject immediately preceded by a “No” response by the subject. A Threshold Criterion is the number of times a Criterion Level must occur to a given level to meet the definition of the Threshold Level.

To obtain accurate hearing test results, the ambient noise of the test environment must be below levels that affect the subject's ability to hear and respond to tonal stimuli. Traditionally, hearing testing by audiologists has been performed in sound-isolating rooms that attenuate ambient noise to levels that do not affect hearing thresholds. Testing in other environments increases access and decreases the cost of hearing testing. Circumaural earphones have been used for decades to increase ambient noise attenuation and are becoming widely used for audiometry, which makes accurate hearing testing possible in a wide variety of environments. The COVID-19 pandemic has added urgency to the need to test in other environments.

### SUMMARY

An audiometric system tests hearing of a subject in an environment in which ambient noise is present. An audiometer tests hearing of the subject by presenting pure tone stimuli to the subject and determining, based on responses received from the subject, a threshold level for each of a set of pure tone stimuli frequencies. An acoustic sensor monitors ambient noise in the vicinity of the subject while the hearing testing of the subject is taking place. A computer receives the threshold levels from the audiometer and sensed ambient noise from the acoustic sensor. The computer determines whether the ambient noise created a distraction to the subject during the determining by the audiometer of any of the threshold levels. The computer generates a test report that displays the threshold levels based on responses from the subject and identifies those threshold levels that require retesting due to presence of their distraction created by ambient noise.

An audiometric system tests hearing of a subject in an environment in which ambient noise is present. An audiometer tests hearing of the subject by presenting pure tone stimuli to the subject and determining, based on responses received from the subject, a threshold level for each of a set of pure tone stimuli frequencies. An acoustic sensor monitors ambient noise in the vicinity of the subject while the hearing testing of the subject is taking place. A computer receives the threshold levels from the audiometer and sensed ambient noise from the acoustic sensor. The computer determines that circumaural earphones were used for testing hearing of the subject and generates a circumaural earphone alert indicating that masking has occurred based upon a Maximum Permissible Ambient Noise Level (MPANL) that is adjusted to include additional attenuation of noise by circumaural earphones.

An audiometric system tests hearing of a subject in an environment in which ambient noise is present. An audiometer tests hearing of the subject by presenting pure tone stimuli to the subject and determining, based on responses received from the subject, a threshold level for each of a set of pure tone stimuli frequencies. An acoustic sensor monitors ambient noise in the vicinity of the subject while the hearing testing of the subject is taking place. A computer receives the threshold levels from the audiometer and sensed ambient noise from the acoustic sensor. The computer generates an alert when a Maximum Permissible Ambient Noise Level (MPANL) that has been adjusted to a modified minimum signal level that is different from 0 dB HL, and the ambient noise level exceeds the modified minimum signal level.

### BRIEF DESCRIPTION OF THE DRAWINGS

FIG. 1 is a block diagram of an audiometric system that includes an ambient noise monitor that produces masking alerts and distraction alerts based on ambient noise present during pure-tone testing.

FIG. 2 is a graph showing a distracting noise spectrum with an insert showing a waveform.

FIG. 3 is a graph showing distribution of crest factors in one second (1-s) segments of the distracting noise.

FIG. 4 is a graph showing time per trial (in seconds) as a function of noise level in an AMTAS test.

FIG. 5 is a graph showing number of trials to achieve threshold as a function of noise level.

FIG. 6 is a graph showing average threshold shift as a function of noise level.

FIG. 7 is a graph showing mean distractibility as a function of noise level.

### DETAILED DESCRIPTION

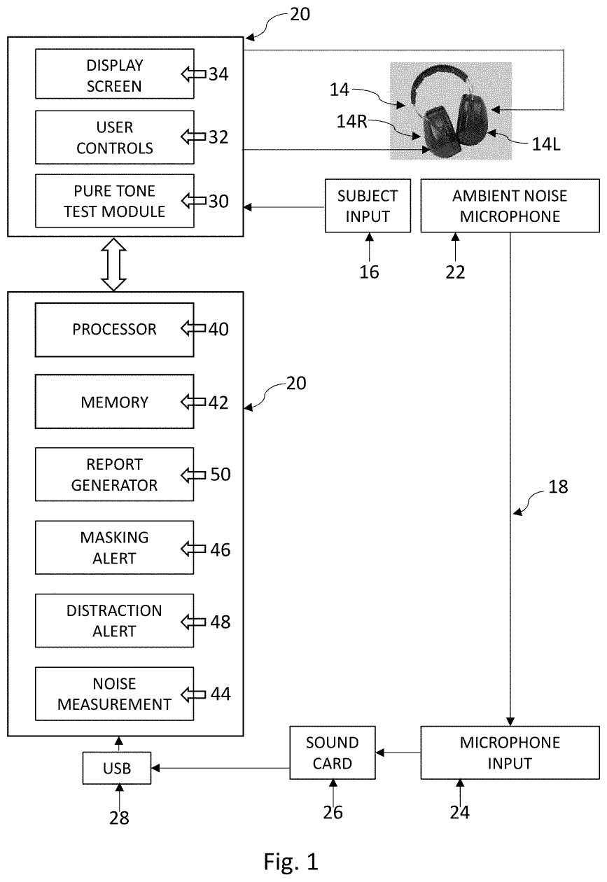

FIG. 1 is a block diagram of audiometric system 10, which performs pure-tone threshold testing in environments in which ambient noise can cause both masking of test tones and distraction of the test subject, and which provides a hearing test report that identifies those pure tone Threshold Levels that are affected by masking effects and by or by distraction effects produced by ambient noise. Audiometric system 10 includes audiometer 12, earphones 14 (including right earphone 14R and left earphone 14L), patient input interface 16, and ambient noise monitor 18 (which includes computer 20, microphone 22, microphone input 24, sound card 26, and universal serial bus (USB) connector 28). Audiometer 12 includes pure-tone test module 30, user controls 32, and display screen 34. Computer 20 includes processor 40, memory 42, noise measurement module 44, masking alert module 46, distraction alert module 48, and report generator 50.

Audiometer 12 is an audiometer that is capable of selectively presenting pure tone stimuli to right earphone 14R and left earphone 14L of earphones 14. Stimuli can be delivered to one earphone at a time, with the other ear receiving masking noise. Audiometer 12 includes a pure tone test module 30, which produces pure tone stimuli at discrete single frequencies in a range from, for example, 250 Hz to 8 kHz or 250 Hz to 16 kHz. Audiometer 12 also controls the intensity of the stimuli over a range of, for example 0 dB to 100 dB. User controls 32 can include keys or buttons and other input devices that allow the examiner conducting the testing to provide instructions and commands for controlling operation of audiometer 12. Display screen 34 provides messages and displays that assist the examiner in performing the testing and in viewing test results. Examples of audiometers that may be used in audiometry system 10 include the GSI AUDIOSTAR PRO, GSI PELLO, and GSI Flex AMTAS audiometers from Grason-Stadler. Examples of earphones include supra-aural earphones (e.g. Telephonics THD-49 with supra-aural cushions) and circumaural earphones (e.g. Radioear DD450, Radioear DD65v2).

During a hearing test, ambient noise microphone 22 is positioned in the the vicinity of the subject, so that the ambient noise being sensed is representative of the ambient noise present near the subject and near earphones 14. For example, microphone 22 may be clipped to clothing that the subject is wearing.

The ambient noise signal from ambient noise microphone 22 is supplied through microphone input 24 and processed by sound card 26 to produce a digitized ambient noise signal that is delivered through USB port 28 to computer 20. The digitized ambient noise signal is time stamped so that it can be synchronized with test results from the testing being performed by audiometer 12.

Audiometer 12 includes pure tone test module 30 that performs the testing by presenting pure tone acoustic stimuli to earphone 14L or 14R and collecting responses from subject input 16 that indicate whether the subject heard or did not hear the pure tone acoustic stimuli. The sequence of stimuli present can be varied depending on the response by the subject. The varying of the sequence can be selected by the examiner that is administering the test, or can be selected automatically by the pure tone test module 30.

User controls 32 allow the examiner to select the parameters to be used to define the test(s) to be performed. User controls 32 can include switches, keys, touch pads, and other user selection devices.

Display screen 34 provides an ability for audiometer 12 to provide instructions, prompts, and test reports to the examiner. In some embodiments, audiometer may also include a port that allows audiometer 12 to send test reports to a printer. Alternatively, test reports can be displayed or sent to a printer or other device by computer 20.

Computer 20 receives digitized ambient noise data through USB port 28 and pure tone test data from audiometer 12. Computer 20 also provides test reports and associated masking alerts and distraction alerts to audiometer 12, so that the examiner that is administering the testing can view the test report and associated masking alerts and distraction alerts. This allows the examiner to see which tests need to be re-run because either masking by the ambient noise prevented the subject from hearing the stimuli, or because the ambient noise distracted the subject.

Computer 20 includes processor 40 (which can be a single processor or multiple processors) and memory 42 (which stores data and program instructions for performing the functions provided by noise measurement module 44, masking alert module 46, distortion alert 48, and report generator 50). Based upon the threshold levels from audiometer 12 and the digitized sensed ambient noise measurements from noise measurement module 44, a test report is generated by report generator 50 in conjunction with masking alert 46 and distraction alert 48. In one embodiment, computer 20 identifies threshold levels that require retesting due to presence of distraction created by ambient noise when 50% or greater of peak levels in one second (1-s) segments exceed 72.5 dB SPL.

Audiometric system 10 monitors the level and characteristics of ambient noise in the test environment during pure tone audiometry. With the use of appropriate algorithms, the possible influence of the ambient noise on the threshold hearing measurements can be determined. There are two contaminating effects of ambient noise on hearing test results—masking and distraction.

The masking effects of ambient noise are governed by known mechanisms, studied extensively in the psychoacoustics literature, by which the threshold of a pure-tone test signal is shifted by ambient noise that is in the spectral region of the signal. Ambient noise levels that mask audiometric test tones have been calculated and incorporated into ANSI S3.1 Maximum Permissible Ambient Noise Levels for Audiometric Test Rooms. The Maximum Permissible Ambient Noise Levels (MPANLs) in the standard pertain to supra-aural earphones, insert earphones, and the ears uncovered condition and assume a test signal of 0 dB HL. Some audiometric systems monitor ambient noise and report potential errors due to masking caused by ambient noise. MPANLs have been calculated for circumaural earphones that are in common use today, and rules for identifying potential masking effects on signal levels higher than 0-dB HL have been derived. These rules are used to derive alerts, using formulas for identifying potential contamination of threshold measurements from ambient noise masking that can be incorporated into audiometer software.

Unlike the masking effect, the distracting effect of ambient noise on pure-tone thresholds has not been systematically studied. This application presents the results of a study of the effects of distracting noise on hearing thresholds in adult listeners, and a guideline for determining the presence of distracting noise that potentially shifts auditory thresholds.

When the frequency content of ambient noise spans the frequency of the test signal, and when the level of the ambient noise exceeds the minimum level required to mask the signal, the threshold can be elevated by an amount that is directly proportion to the noise level. The minimum ambient noise level that masks a pure-tone presented at 0 dB HL is termed Maximum Permissible Ambient Noise Level (MPANL; ANSI S3.1-1999). The amount of threshold shift can be predicted based on established relations between noise levels and masked thresholds. When the noise contains energy at the test frequency (i.e. direct masking), there is a 1:1 relation between the noise level and the masked threshold. For every 1-dB change of the noise level, the threshold is shifted by 1 dB. The amount of masking is determined by a) the ambient noise level, b) the attenuation provided by the earphone, and c) the listener's hearing threshold (i.e., the level of the test signal at threshold). The MPANLs provided in the standards (discussed below) assume a supra-aural earphone and a test signal of 0 dB HL. Modified MPANLs are provided that take into account additional attenuation provided by circumaural earphones and the listener's hearing sensitivity.

Two standards provide guidance for controlling ambient noise during hearing testing—Occupational Safety and Health standard 1910.95, Appendix D (OSHA, 2008) and American National Standards Institute S3.1 (ANSI, 1999). The use of supra-aural earphones (e.g. Telephonics TDH-49 with supra-aural cushions) is assumed in both standards. The OSHA standard provides MPANLs as octave band levels. The ANSI standard provides both octave band and ⅓ octave band levels.

Relative to the attenuation provided by supra-aural earphones, circumaural earphones (e.g. Radioear DD450, Radioear DD65v2) provide additional attenuation of the ambient noise level that reaches the ear, reducing the masking effect. The additional attenuation permits higher ambient noise level before masking occurs—higher MPANLs. In this section, MPANLs that take into account the higher ambient noise attenuation provided by circumaural earphones are derived.

MPANLs for circumaural earphones were calculated by adding the additional attenuation produced by the circumaural cushions relative to the attenuation produced by supra-aural earphones. MPANLs for three circumaural earphones were calculated with the following formula:

MPANL(C)=MPANL(S)+(AS−AC);

where MPANL(C) is the derived Maximum Permissible Ambient Noise Level for the circumaural earphone; MPANL(S) is the Maximum Permissible Ambient Noise Level for a supra-aural earphone from the standard (ANSI S3.1-1999); AS is the ambient noise attenuation provided by the supra-aural earphone (ANSI S 3.1-1999, Table A.1); and AC is the ambient noise attenuation provided by the circumaural earphone.

From the MPANLs and modified MPANLs, algorithms can be developed that represent rules for alerting the examiner that contamination by ambient noise may have occurred. These algorithms are referred to as alerts. Examiners and manufacturers can implement the alerts based on the needs of the testing facility. In some cases an alert may trigger an automatic retest of a particular threshold to obtain a result when the noise level is lower. These alerts include Standard Alerts, Circumaural Earphone Alerts, Standard Alerts Modified for Minimum Signal Level, Standard Alerts Modified for Threshold Level, and Uncovered Ear Alerts.

Standard Alerts, the simplest form of alerts, occur when the ambient noise level exceeds the MPANL from the standard, typically the OSHA standard for industrial testing and the ANSI standard for clinical testing. With these alerts, the tester is notified when the noise level exceeds the MPANL at any frequency at any time during the test. The standards do not provide guidance on what should be done when one or more MPANL is exceeded. They imply that if an MPANL is exceeded any time during the test, the results are invalid.

Circumaural Earphone Alerts take into account the additional attenuation of ambient noise provided by circumaural earphones relative to supra-aural earphones. The additional attenuation reduces the masking effects of ambient noise so that higher MPANLs are appropriate. Circumaural Earphone Alerts are calculated by adding the additional attenuation provided by circumaural earphones to the MPANLs provided in the standards for supra-aural earphones.

Standard Alerts Modified for Minimum Signal Level are based upon recognition in ANSI S3.1 (1999) that it may be desirable for the minimum signal level to be higher or lower than 0 dB HL. The Standard specifies that MPANLs should be adjusted appropriately when hearing thresholds for pure tones are measured above and below 0 dB HL. These alerts are adjusted to take into account the lowest level at which the tester wishes to measure thresholds. A minimum level that is greater than 0 dB HL increases the allowable ambient noise level. An alert occurs when a MPANL is exceeded any time during the test.

Standard Alerts Modified for Threshold Level take into account the threshold level of the listener. A hearing-impaired listener who has thresholds higher than 0 dB HL are less affected by ambient noise than listeners with better hearing sensitivity. A listener with a threshold of 50 dB, for example, would not be affected by ambient noise until the noise level is 50 dB above the standard MPANL. Standard Alerts Modified for Threshold Level are frequency and ear specific so that individual thresholds can be identified that may have been affected by ambient noise. In some cases one or two thresholds may have been affected but the rest of the thresholds are not susceptible to contamination by the ambient noise.

Uncovered Ear Alerts are important for testing bone-conduction thresholds when the test ear is not covered by an earphone. This is the condition that is most vulnerable to contamination by ambient noise because there is no earphone attenuation to reduce the effects of the noise. The OSHA standard is concerned with industrial air-conduction testing and not bone-conduction testing, which is rarely performed in industrial hearing conservation programs. Accordingly, the OSHA standard does not provide MPANLs for the uncovered ear. Uncovered Ear Alerts are presented for three situations—no correction (standard MPANLs), correction for a Minimum Test Level that is higher than 0 dB HL, and correction for the threshold of the listener at each frequency.

A second influence of ambient noise on hearing test results is distraction. Although there has been very little research on the influence of distractions on hearing test results, it is possible that ambient noise elevates and increases the variability of measured thresholds independently of the masking effect. An experiment was conducted to measure distraction masking effects on auditory thresholds measured by an automated pure-tone audiometry method (AMTAS) in adult listeners.

A distracting noise was obtained from a public collection of sound files (https://freesound.org/people/flppnchnnlz/sounds/505529/). The recording was made in a warehouse during a workday. It is described as follows: “the lighting fixtures buzz and overall walla. Some banging of totes and boxes, laughing voices, general warehouse standard”.

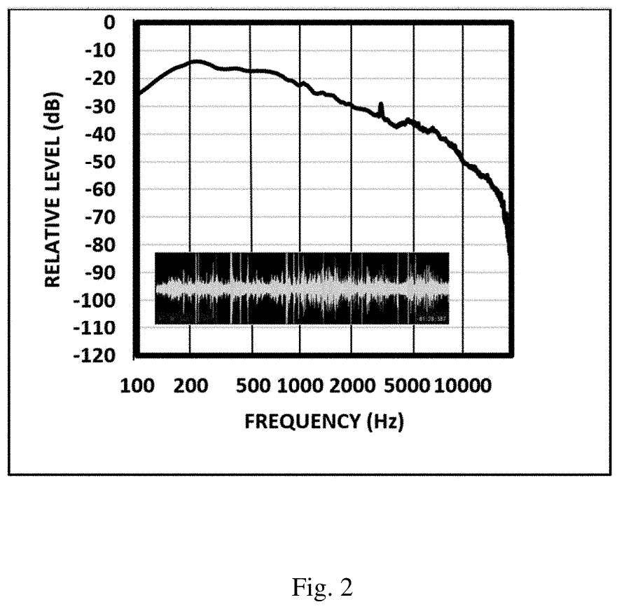

The waveform and spectrum are shown in FIG. 2. Waveform statistics (peak and rms) and spectra were measured with Adobe Audition software based on the digitized waveform sampled at a rate of 44,100 Hz. The sound has a steady background with superimposed transients from equipment, voices, and other warehouse activity. The spectral peak occurs at 200 Hz and rolls off at −5 dB/octave to 8 kHz and more sharply beyond that frequency. The duration of the noise signal is 111 s.

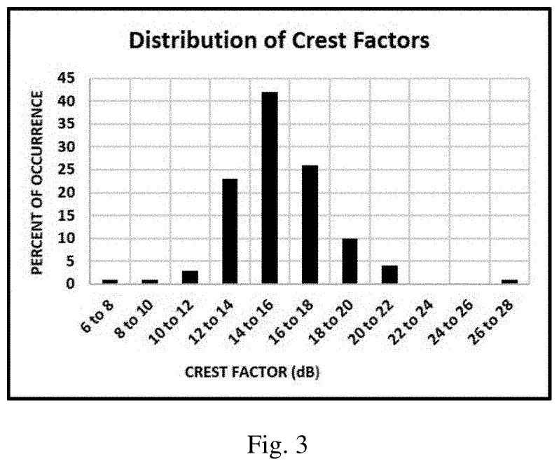

An analysis of all the (1-s) segments of the signal shows the distribution of crest factors (peak to rms ratio) shown in FIG. 3. The overall crest factor was 21 dB. The median crest factor in all 111 1-s segments was 15 dB. Crest factor is an important metric for distracting noise because it is probably the peak levels that produce distraction.

Air-conduction audiograms were obtained from ten ears of five subjects (age 30-74 years) with an automated test system (GSI Flex AMTAS) with Sennheiser HDA 200 circumaural earphones, calibrated in compliance with ANSI S3.6-2018. AMTAS acquires pure-tone thresholds with a modified Hughson-Westlake method that has been validated against audiograms obtained by expert audiologists. The system logs each stimulus and response so the number of stimulus presentations (trials) can be recovered for each threshold determination. Subjects were self-reported to have no significant hearing difficulty or history of ear disease. Seven audiograms were obtained from each subject (baseline without distracting noise and six with distracting noise). The first audiogram consisted of thresholds at six test frequencies (250-8000 Hz octave frequencies). Average thresholds of the five subjects are shown in Table 1:

| | Frequency (Hz) | | | | | |
|---|---|---|---|---|---|---|
| | 250 | 500 | 1000 | 2000 | 4000 | 8000 |
| Mean (dB HL) | 10 | 10 | 9 | 11 | 20 | 22 |
| SD (dB) | 11 | 7 | 12 | 9 | 19 | 26 |

Subsequent audiograms were obtained at three test frequencies (500, 1000, 2000 Hz). The restricted range of test frequencies was selected to limit the total duration of the listening session to about 45 minutes and was deemed adequate to explore the distracting effect of the noise. During these tests the distracting noise was played from a loudspeaker controlled by a second computer. The 111-s noise signal was looped continuously during the test. The loudspeaker was placed directly in front of the listener at a distance of 81 cm. The distracting noise was presented at 40, 50, 60, 70, 80, and 90 dB SPL, measured with the microphone of a sound level meter (Larson Davis System 824) placed 2.5 cm from the listener's chest. The order of distracting noise levels was randomized. Testing was performed in a quiet office with no nearby noise sources. Third-octave ambient noise levels were 15-30 dB below the MPANLs for DD450 earphones. The DD450 earphone is equivalent to the HDA 200 earphone.

The effects of distracting noise were measured with the following metrics:

Average time per trial—The trial time consists of a ready interval, a vote interval, the subjects' response time, and a delay of approximately 1 s before the beginning of the next trial. The trial count includes all stimuli presented for a threshold measurement.

Average number of trials required to measure threshold—For each threshold determination the starting level is 40-dB HL. The listener votes Yes or No after each stimulus presentation by touching a button on the touch screen placed directly in front of the listener. The level descends in 10-dB steps until a No response occurs, after which the level increases in 5-dB steps until a Yes response occurs. The level then decreases by 5 dB until a No response occurs, and then increases by 5 dB until a Yes response occurs. This process continues until two Yes responses preceded by No responses occur in a 5-dB window.

Threshold shift—Threshold shift is the difference in thresholds between those obtained while the distracting noise is on and the baseline audiogram obtained with no distracting noise (threshold in noise—threshold in quiet).

Perceived distractibility of the noise—Perceived distractibility was measured on a three-point scale after each audiogram. The listener was asked to rate the distractibility of the noise as follows.

Rating—

0—Not distracting—I was able to concentrate on the tones and respond correctly.

1—Somewhat distracting—The background noise occasionally interfered with my ability to concentrate on the tones.

2—Very distracting—I was not able to focus on the tones during the test.

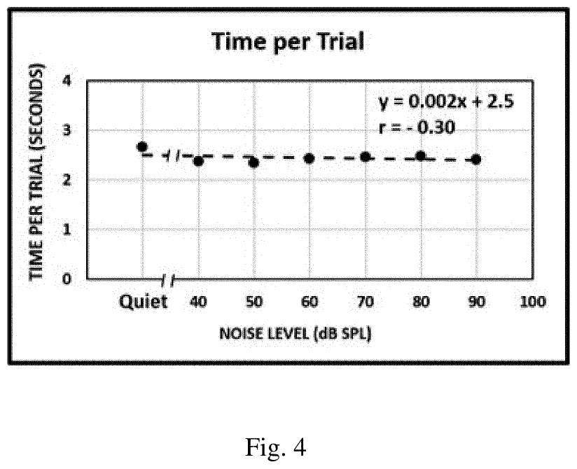

Average time per trial for the quiet condition and noise conditions is shown in FIG. 4. Time per trial was not affected by the distracting noise. The average time per trial was 2.45 s across all noise levels.

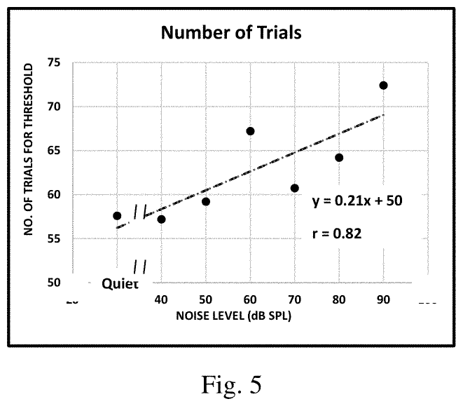

Number of trials to reach threshold—The average number of trials required to measure threshold increased linearly with the level of the distracting noise (FIG. 5). Each 10-dB increase in the noise level resulted in an increase of three (3) trials to achieve threshold. The correlation coefficient was 0.92 indicating a strong relation between number of trials and noise level.

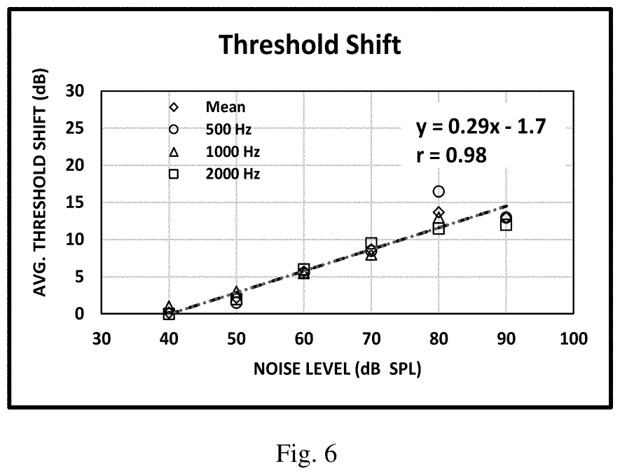

Mean threshold shift for the six noise levels relative to the threshold in quiet are shown in FIG. 6 for each test frequency. Analysis of variance indicated that the effect of noise level was statistically significant (p<0.01) and the effect of stimulus frequency was not significant (p>0.05). Each 10-dB increase in the noise level resulted in a 3.3-dB increase in average thresholds. The correlation coefficient between mean threshold shift and noise level was 0.94 indicating a strong relation between threshold shift and noise level. Determined by interpolation, a noise level of 57.5 dB SPL produces an average threshold shift of 5 dB. This level corresponds to a median peak level of 72.5 dB SPL (rms noise level plus crest factor). The level producing a 5-dB threshold shift was selected because it represents one intensity level step during routine audiometry.

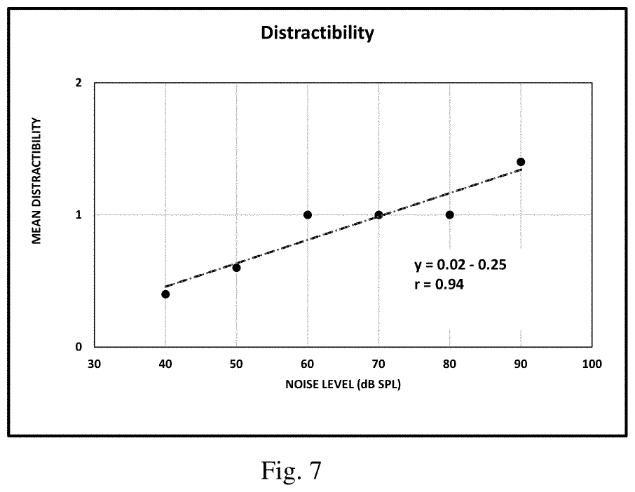

As shown in FIG. 7, mean distractibility ratings increased linearly with increasing noise level. The correlation coefficient was 0.98 indicating a strong relation between distractibility ratings and noise level.

The first part of this discussion provides rules for noise alerts based on MPANLs that are in OSHA and ANSI standards. The values in those standards assume a supra-aural earphone with its limited ambient noise attenuation and a signal level of 0-dB HL. Circumaural earphones available for audiometry provide greater ambient noise attenuation and reduce the contaminating effects of ambient noise. When the signal level is higher than 0-dB HL, such as with subjects with hearing loss, the contaminating effects of ambient noise are reduced. Noise alert rules are provided that take into account the additional attenuation provided by circumaural earphones and the threshold levels of the listener. Future standards should incorporate other earphones and other signal levels into the MPANL values.

The results of the distractibility experiment reveal a contaminating effect of ambient noise on hearing test results that is different from the direct masking effects of steady-state noise. The distracting noise had no effect on response time per trial (FIG. 4). However, the number of trials required to achieve threshold increased with noise level at a rate of 3 trials for each increase of 10 dB of the distracting noise (FIG. 5). The increase in number of trials for threshold is the opposite of the expected effect of the upward threshold shift shown in FIG. 6. A higher threshold would be expected to result in fewer trials needed for threshold because of the proximity of threshold to the starting level (40-dB HL). The increase in the number of trials is likely an indication of distraction, resulting in less consistency in responses requiring more trials to meet the threshold criterion, the phenomenon described as distraction masking.

The threshold shifts produced by the distracting noise behave differently than those that occur from direct masking. Threshold shifts from direct masking increase at a rate of 1.0 dB/dB as masking level increases. The threshold shifts observed here resulting from distracting noise increase at a rate of 0.29 dB/dB (FIG. 6), clearly different from the 1:1 increase in thresholds expected from direct masking. In addition, the observation that threshold shifts produced by the distracting noise were the same at all test frequencies (FIG. 6) indicates that the shifts were not due to direct masking. The ⅓ octave level of the distracting noise is 12 dB lower at 2 kHz compared to 1 kHz, and yet the threshold shifts were not significantly different at the two frequencies. Direct masking would lead to an expectation of 12 dB greater threshold shift at 1 kHz.

These results suggest an additional effect of ambient noise on thresholds when the noise has temporal properties that are common in workspaces and vary in time like the waveform depicted in FIG. 2. The crest factor of the distracting noise (21 dB) is substantially higher than wide-band noise and speech (˜12 dB). The irregularly occurring peaks in the distracting noise may produce the distractions that result in increased trials for threshold and threshold shifts.

The results shown in FIG. 6 indicate that a noise level of 57.5 dB SPL corresponds to a 5 dB threshold shift. That level corresponds to a median peak level of 72.5 dB (SPL plus 15-dB crest factor). This suggests the following rule for notifying the tester of a possible contaminating influence of background noise that is separate from the MPLANL considerations discussed for masking alerts: A Distraction Alert is reported when 50% or greater of the peak levels in 1-s segments exceed 72.5 dB SPL.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A system for testing hearing of a subject in an environment in which ambient noise is present, the system comprising:
an audiometer for testing hearing of the subject by presenting pure tone acoustic stimuli to the subject and determining, based on responses received from the subject, a threshold level for each of a set of pure tone stimuli frequencies;
an acoustic sensor for sensing ambient noise in a vicinity of the subject while the testing hearing of the subject is taking place;
a computer that receives the threshold levels from the audiometer and ambient noise sensed by the acoustic sensor, determines whether the ambient noise created a distraction to the subject during the determining by the audiometer of any of the threshold levels, and generates a test report displaying the threshold levels based on responses from the subject and identifying those threshold levels that require retesting due to presence of the distraction created by ambient noise.

2. The system of claim 1 wherein the computer identifies threshold levels that require retesting due to presence of distraction created by ambient noise when 50% or greater of peak levels in one second (1-s) segments exceed 72.5 dB SPL.

3. The system of claim 1, wherein the computer determines that circumaural earphones were used for testing hearing of the subject and generates a circumaural earphone alert indicating that masking has occurred based upon a Maximum Permissible Ambient Noise Level (MPANL) that is adjusted to include additional attenuation of noise by circumaural earphones.

4. A system for testing hearing of a subject in an environment in which ambient noise is present, the system comprising:
an audiometer for testing hearing of the subject by presenting, sequentially, test stimuli at pre-determined frequencies to the subject; receiving responses from the subject to the stimuli; identifying a threshold level for each test tone frequency for the first ear based on the responses;
an acoustic sensor for sensing ambient noise in a vicinity of the subject while the testing hearing of the subject is taking place;
a computer that receives the threshold levels from the audiometer and sensed ambient noise from the acoustic sensor, determines whether the ambient noise created a distraction to the subject during the measuring by the audiometer of way of the test stimuli, and generates a test report displaying the threshold levels based on responses from the subject and identifying those threshold levels that require retesting due to presence of their distraction created by ambient noise.

5. The system of claim 3 wherein the computer identifies threshold levels that require retesting due to presence of distraction created by ambient noise when 50% or greater of peak levels in one second (1-s) segments exceed 72.5 dB SPL.

6. The system of claim 4, wherein the computer determines whether circumaural earphones were used for testing the subject, and determines whether the ambient noise during testing by the audiometer exceeded an adjusted Maximum Permissible Ambient Noise Level (MPANL) that is a function of attenuation of ambient by circumaural earphones, and generates a test report displaying the threshold levels based on responses from the subject and identifying those threshold levels that require retesting due to presence of masking by the ambient noise.

7. A method of testing hearing of a subject in an environment in which ambient noise is present, the method comprising:
presenting pure tone acoustic stimuli to the subject;
collecting responses from the subject that indicate whether the subject has heard the pure tone acoustic stimuli;
identifying threshold levels at which the subject hears pure tone acoustic stimuli of different frequencies based on the response from the subject;
sensing ambient noise present while the acoustic test stimuli are being presented to the subject;
identifying responses from the subject that correspond to time periods during which the ambient noise sensed produces a distraction to the subject that negatively affect accuracy of the responses by the subject; and
presenting a test report that displays the threshold levels at different frequencies based upon the response from the subject, and providing distraction alerts that identify those threshold levels that require retesting based upon presence of distraction produced by ambient noise when those threshold levels were being tested.

8. The method of claim 7 and further comprising, identifying ambient noise that produces a distraction when 50% or greater of peak levels in 1 second segments exceed 72.5 dB SPL.

9. The system of claim 7, wherein the determines that circumaural earphones were used for testing hearing of the subject and generates a circumaural earphone alert indicating that masking has occurred based upon a Maximum Permissible Ambient Noise Level (MPANL) that is adjusted to include additional attenuation of noise by circumaural earphones.

10. A method of testing hearing of a subject in an environment in which ambient noise is present, the method comprising:
providing, to the subject, a sequence of pure tone acoustic stimuli of different frequencies;
receiving, from the subject, responses to the sequence of pure tone acoustic stimuli;
adaptively selecting the acoustic pure tone stimuli based upon the subject's responses;
identifying hearing threshold levels based on the subject's responses;
sensing ambient noise present in a vicinity of the subject during testing or hearing of the subject;
displaying a diagnostic audiogram based upon the hearing threshold level of the subject of each of set of different pure tone stimuli frequencies; and
displaying as part of the diagnostic audiogram, a distraction alerts that identify hearing threshold levels requiring retesting based upon levels of the sensed ambient noise at time periods during which pure tone acoustic stimuli are being provided to the subject.

11. The method of claim 10 and further comprising, identifying ambient noise that produces a distraction when 50% or greater of peak levels in 1 second segments exceed 72.5 dB SPL.

12. The system of claim 10, wherein the computer determines whether circumaural earphones were used for testing the subject, and determines whether the ambient noise during testing by the audiometer exceeded an adjusted Maximum Permissible Ambient Noise Level (MPANL) that is a function of attenuation of ambient by circumaural earphones, and generates a test report displaying the threshold levels based on responses from the subject and identifying those threshold levels that require retesting due to presence of masking by the ambient noise.

13. A system for testing hearing of a subject in an environment in which ambient noise is present, the system comprising:
an audiometer for testing hearing of the subject by presenting pure tone acoustic stimuli to the subject and determining, based on responses received from the subject, a threshold level for each of a set of pure tone stimuli frequencies;
an acoustic sensor for sensing ambient noise in a vicinity of the subject while the testing hearing of the subject is taking place;
a computer that receives the threshold levels from the audiometer and the acoustic sensor, wherein the computer determines that circumaural earphones were used for testing hearing of the subject and generates a circumaural earphone alert indicating that masking has occurred based upon a Maximum Permissible Ambient Noise Level (MPANL) that is adjusted to include additional attenuation of noise by circumaural earphones.

14. A system for testing hearing of a subject in an environment in which ambient noise is present, the system comprising:
an audiometer for testing hearing of the subject by presenting, sequentially, test stimuli at pre-determined frequencies to the subject, receiving responses from the subject to the stimuli, and identifying a threshold level for each test tone frequency for the first ear based on the responses;
an acoustic sensor for sensing ambient noise in a vicinity of the subject while the testing hearing of the subject is taking place;
a computer that receives the threshold levels from the audiometer and sensed ambient noise from the acoustic sensor, determines whether circumaural earphones were used for testing the subject, and determines whether the ambient noise during testing by the audiometer exceeded an adjusted Maximum Permissible Ambient Noise Level (MPANL) that is a function of attenuation of ambient by circumaural earphones, and generates a test report displaying the threshold levels based on responses from the subject and identifying those threshold levels that require retesting due to presence of masking by the ambient noise.

15. A method of testing hearing of a subject in an environment in which ambient noise is present, the method comprising:
presenting pure tone acoustic stimuli to the subject;
collecting responses from the subject that indicate whether the subject has heard the pure tone acoustic stimuli;
identifying threshold levels at which the subject hears pure tone acoustic stimuli of different frequencies based on the response from the subject;
sensing ambient noise present while the acoustic test stimuli are being presented to the subject;
displaying a diagnostic audiogram based upon the hearing threshold level of the subject of each of set of different pure tone stimuli frequencies; and
determining whether circumaural earphones were used for testing the subject;
determining whether the ambient noise during testing by the audiometer exceeded an adjusted Maximum Permissible Ambient Noise Level (MPANL) that is a function of attenuation of ambient by circumaural earphones; and
displaying, as part of the diagnostic audiogram, alerts that identify hearing threshold levels requiring retesting based upon levels of the sensed ambient noise exceeded the adjusted MPANL.

16. A method of testing hearing of a subject in an environment in which ambient noise is present the method comprising:
providing, to the subject, a sequence of pure tone acoustic stimuli of different frequencies;
receiving, from the subject, response to the sequence of pure tone acoustic stimuli;
adaptively selecting the acoustic pure tone stimuli based upon the subject's responses;
identifying hearing threshold levels based on the subject's responses;
sensing ambient noise present in a vicinity of the subject during testing or hearing of the subject;
displaying a diagnostic audiogram based upon the hearing threshold level of the subject of each of set of different pure tone stimuli frequencies;
determining whether circumaural earphones were used for testing the subject;
determining whether the ambient noise during testing by the audiometer exceeded an adjusted Maximum Permissible Ambient Noise Level (MPANL) that is a function of attenuation of ambient by circumaural earphones; and
displaying, as part of the diagnostic audiogram, an alert that identifies hearing threshold levels requiring retesting based upon levels of the sensed ambient noise exceeded the adjusted MPANL.
displaying, as part of the diagnostic audiogram, alerts that identify hearing threshold levels requiring retesting based upon levels of the sensed ambient noise exceeded the adjusted MPANL.

17. A system for testing hearing of a subject in an environment in which ambient noise is present, the system comprising:
an audiometer for testing hearing of the subject by presenting pure tone acoustic stimuli to the subject and determining, based on responses received from the subject, a threshold level for each of a set of pure tone stimuli frequencies;
an acoustic sensor for sensing ambient noise in a vicinity of the subject while the testing hearing of the subject is taking place;
a computer that receives the threshold levels from the audiometer and sensed ambient noise the acoustic sensor, wherein the computer generates an alert when a Maximum Permissible Ambient Noise Level (MPANL) that has been adjusted to a modified minimum signal level that is different from 0 dB HL, and the ambient noise level exceeds the modified minimum signal level.

18. A system for testing hearing of a subject in an environment in which ambient noise is present, the system comprising:
an audiometer for testing hearing of the subject by presenting, sequentially, test stimuli at pre-determined frequencies to the subject, receiving responses from the subject to the stimuli, and identifying a threshold level for each test tone frequency for the first ear based on the responses;
an acoustic sensor for sensing ambient noise in a vicinity of the subject while the testing hearing of the subject is taking place;
a computer that receives the threshold levels from the audiometer and sensed ambient noise from the acoustic sensor, and generates a test report displaying the threshold levels based on responses from the subject and identifying those threshold levels that require retesting due to presence of masking by the ambient noise, wherein the computer generates a masking alert when a hearing impaired subject has an adjusted threshold that is her than 0 dB HL and the sensed ambient noise exceeds the adjusted threshold that is higher than the adjusted that is higher than the sensed ambient noise.

19. A method of testing hearing of a subject in an environment in which ambient noise is present, the method comprising:
presenting pure tone acoustic stimuli to the subject;
collecting responses from the subject that indicate whether the subject has heard the pure tone acoustic stimuli;
identifying threshold levels at which the subject hears pure tone acoustic stimuli of different frequencies based on the response from the subject;
sensing ambient noise present while the acoustic test stimuli are being represented to the subject;
identifying responses from the subject that correspond to time periods during which the ambient noise sensed produces a distraction to the subject that negatively affect accuracy of the responses by the subject; and
presenting a test report that displays the threshold levels at different frequencies based upon the response from the subject, and providing alerts that identify those threshold levels that require retesting based upon presence of masking produced by ambient noise when those threshold levels were being tested, wherein an alert is generated when a Maximum Permissible Ambient Noise Level (MPANL) has been adjusted to a modified minimum signal level that is different from 0 dB HL, and the ambient noise level exceeds the modified minimum signal level.

20. A method of testing hearing of a subject in an environment in which ambient noise is present the method comprising:
providing, to the subject, a sequence of pure tone acoustic stimuli of different frequencies;
receiving, from the subject, response to the sequence of pure tone acoustic stimuli;
adaptively selecting the acoustic pure tone stimuli based upon the subject's responses;
identifying hearing threshold levels based on the subject's responses;
sensing ambient noise present in a vicinity of the subject during testing or hearing of the subject;
displaying a diagnostic audiogram based upon the hearing threshold level of the subject of each of set of different pure tone stimuli frequencies, wherein a masking alert is generated as part of the diagnostic audiogram when a hearing impaired subject has an adjusted threshold that is greater than 0 dB HL and the sensed ambient noise exceeds the adjusted threshold.
